(19) United States
(12) Patent Application Publication
(10) Pub. No.: US 2025/0115844 A1
(43) Pub. Date: Apr. 10, 2025

(54) CELL CULTURE APPARATUS AND CELL CULTURE SYSTEM

(71) Applicants: CANON MEDICAL SYSTEMS CORPORATION, Tochigi (JP); Canon Kabushiki Kaisha, Tokyo (JP)

(72) Inventors: Takahiro ISHII, Utsunomiya (JP); Kazutaka YANAGITA, Yokohama (JP); Noritaka UKIYO, Tokyo (JP)

(73) Assignees: CANON MEDICAL SYSTEMS CORPORATION, Tochigi (JP); Canon Kabushiki Kaisha, Tokyo (JP)

(21) Appl. No.: 18/907,249

(22) Filed: Oct. 4, 2024

(30) Foreign Application Priority Data
Oct. 6, 2023 (JP) 2023-174557

(51) Int. Cl. C12M 3/00; C12M 1/00

(52) U.S. Cl. CPC C12M 23/42; C12M 23/38; C12M 23/48

(57) ABSTRACT

A cell culture apparatus includes: a casing that, while a cartridge has a culturing vessel used for culturing cells, forms a chamber space in which at least a part of the cartridge including the culturing vessel is installed; an opening configured to allow communication between the inside and the outside of the chamber space and provided for the purpose of installing said at least a part of the cartridge in the chamber space; a moving equipment configured to move the cartridge, for the purpose of installing or ejecting said at least a part of the cartridge into or from the chamber space via the opening; and an opening cover configured to open the opening in association with a moving operation of the moving equipment to install the cartridge and/or configured to close the opening in association with a moving operation of the moving equipment to eject the cartridge.

### CROSS-REFERENCE TO RELATED APPLICATIONS

This application is based upon and claims the benefit of priority from Japanese Patent Application No. 2023-174557, filed on Oct. 6, 2023; the entire contents of which are incorporated herein by reference.

### FIELD

Embodiments described herein relate generally to a cell culture apparatus and a cell culture system.

### BACKGROUND

Conventionally, cell culture is conducted in an environment set to be most suitable for cells to be cultured. For example, in the culture of induced pluripotent stem cells (iPS cells), culture is conducted in a culture chamber controlled to “temperature: 37° C., humidity: 90%, and CO2 concentration: 5%” in order to maintain a culture medium environment.

### BRIEF DESCRIPTION OF THE DRAWINGS

FIG. 1 is an external view of a cell culture system according to a first embodiment;

FIG. 2 is a drawing for explaining an incubator according to the first embodiment;

FIG. 3 is a drawing for explaining a cartridge according to the first embodiment;

FIG. 4 is a schematic drawing illustrating an exemplary configuration of a cell culture apparatus according to the first embodiment;

FIG. 5A is a schematic drawing illustrating an example of a seal member according to the first embodiment;

FIG. 5B is a schematic drawing illustrating an example of an opening cover according to the first embodiment;

FIG. 6 is a schematic drawing illustrating an exemplary configuration of a cell culture apparatus according to a modification example;

FIG. 7 is a schematic drawing illustrating an exemplary configuration of a cell culture apparatus according to another modification example;

FIG. 8 is a schematic drawing illustrating an exemplary configuration of a cell culture apparatus according to a second embodiment; and

FIG. 9 is a schematic drawing illustrating an example of an opening cover according to the second embodiment.

### DETAILED DESCRIPTION

A cell culture apparatus according to an embodiment includes a casing, an opening, a moving equipment, and an opening cover. While a cartridge has a culturing vessel used for culturing cells, the casing forms a chamber space in which at least a part of the cartridge including the culturing vessel is installed. The opening is configured to allow communication between the inside and the outside of the chamber space and is provided for the purpose of installing said at least a part of the cartridge in the chamber space. The moving equipment is configured to move the cartridge, for the purpose of installing or ejecting said at least a part of the cartridge into or from the chamber space via the opening. The opening cover is configured to open the opening in association with a moving operation of the moving equipment to install the cartridge and/or configured to close the opening in association with a moving operation of the moving equipment to eject the cartridge.

Exemplary embodiments of a cell culture apparatus and a cell culture system of the present disclosure will be explained in detail below, with reference to the accompanying drawings. The cell culture apparatus and the cell culture system of the present disclosure are not limited by the embodiments described below. Further, in the following description, some of the constituent elements that are the same as one another will be referred to by using the same reference characters, and duplicate explanations thereof will be omitted.

### First Embodiment

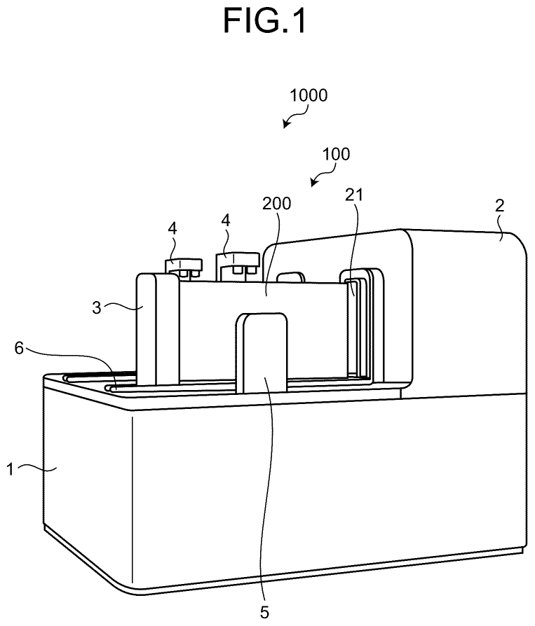

FIG. 1 is an external view of a cell culture system 1000 according to a first embodiment. As illustrated in FIG. 1, the cell culture system 1000 according to the present embodiment includes: a cell culture apparatus 100 including an apparatus base part 1, an incubator 2, a cooling mechanism 3, a liquid delivery mechanism 4, a valve open/close mechanism 5, and a moving equipment 6; and a cartridge 200. The cell culture system 1000 is configured to carry out cell culture by using the cartridge 200.

The cartridge 200 has a culturing vessel used for culturing cells and may be installed within the incubator 2 of the cell culture apparatus 100. Further, the cartridge 200 is provided with a reagent and closed-system flow paths and is configured to be able to perform various types of steps related to the cell culture.

The apparatus base part 1 is configured to hold the incubator 2, the cooling mechanism 3, the liquid delivery mechanism 4, the valve open/close mechanism 5, and the moving equipment 6 and is configured to control the constituent elements. For example, the apparatus base part 1 includes processing circuitry for controlling the abovementioned constituent elements. As a result of the processing circuitry controlling the constituent elements as described below, the entirety of the cell culture apparatus 100 is controlled.

The incubator 2 includes a gas supplier configured to supply gas, a temperature controlling unit configured to control temperatures, various types of sensors, and the like. The incubator 2 is configured to exercise control so that a chamber space formed therein realizes an environment suitable for the cell culture. More specifically, the incubator 2 is configured to exercise control so that the temperature, humidity, CO₂ concentration, and the like of the chamber space are under substantially constant conditions. For example, the incubator 2 is configured to exercise control so that the environment in the chamber space satisfies “temperature: 37° C.; humidity: 90%; and CO₂ concentration: 5%”.

In this situation, as illustrated in FIG. 1, the incubator 2 has an opening 21 that allows communication between the inside and the outside of the chamber space. Via the opening 21, a cartridge 200 is installed in the chamber space. In this situation, the incubator 2 is an example of a casing. The opening 21 is an example of an opening.

The cooling mechanism 3 is configured to cool the reagent held in the cartridge 200.

The liquid delivery mechanism 4 is configured to control liquid delivery in flow paths included in the cartridge 200. More specifically, the liquid delivery mechanism 4 is a pressurizing mechanism configured to apply energy for delivering a cell liquid including cells and the reagent to the flow paths in the cartridge 200. For example, the liquid delivery mechanism 4 is configured to deliver the cell liquid and the reagent to the flow paths in the cartridge 200, by being connected to a connection port provided in the top face of the cartridge 200 and applying the energy to the inside of the cartridge.

The valve open/close mechanism 5 is configured to control a switching valve for switching between the flow paths included in the cartridge 200. More specifically, at various steps of the cell culture performed in the cartridge 200, the valve open/close mechanism 5 is configured to switch between the flow paths in the cartridge 200 by controlling opening and closing of the switching valves provided for the closed-system flow paths, so as to allow the reagent, the cell liquid, and the like used in the various steps to flow through the paths.

The moving equipment 6 is configured to move the cartridge 200 to the chamber space in the incubator 2 through the opening 21. More specifically, the moving equipment 6 includes a drive source such as a motor and a supporter for supporting the cartridge 200 and is configured to linearly move the cartridge 200 by transmitting drive power generated by the drive source to the supporter. For example, the moving equipment 6 is configured to install the cartridge 200 into the incubator by moving the cartridge 200 toward the incubator 2 and is configured to eject the cartridge 200 from the inside of the incubator 2, by moving the cartridge 200 to the outside of the incubator 2. In this situation, the moving equipment 6 is an example of a moving equipment.

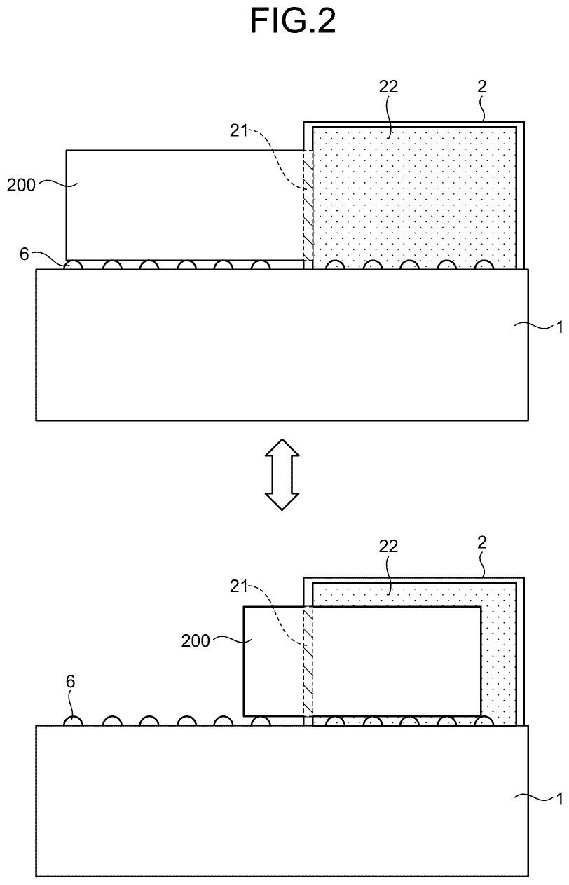

In this situation, in the cell culture apparatus 100 according to the present embodiment, a part of the cartridge 200 is installed in the chamber space of the incubator 2. FIG. 2 is a drawing for explaining the incubator 2 according to the first embodiment. In this situation, FIG. 2 schematically illustrates the cell culture apparatus 100 as viewed from a direction orthogonal to the direction in which the cartridge 200 is moved by the moving equipment 6.

As illustrated in the top section of FIG. 2, the incubator 2 has a chamber space 22 formed therein and has the opening 21 provided on the cartridge 200 side. By the gas supplier, the temperature controlling unit, and the various types of sensors, the incubator 2 is controlled so that ambient air in the chamber space 22 is under a condition (e.g., “temperature: 37° C.; humidity: 90%; and CO₂ concentration: 5%”) suitable for the cell culture.

Further, while the cartridge 200 has the culturing vessel, the incubator 2 is configured so that at least a part of the cartridge 200 including the culturing vessel is installed in the chamber space 22. In other words, as illustrated in the bottom section of FIG. 2, a part of the cartridge 200 is inserted into the chamber space 22 of the incubator 2, at a step of the cell culture. In this situation, the part of the cartridge 200 installed in the chamber space 22 includes the culturing vessel.

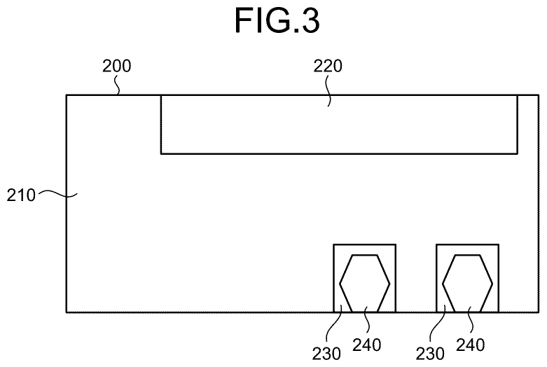

FIG. 3 is a drawing for explaining the cartridge 200 according to the first embodiment. In the present example, FIG. 3 schematically illustrates a partial configuration inside the cartridge 200. As illustrated in FIG. 3, the inside of the cartridge 200 is divided into a plurality of spaces, and various types of elements are provided therein. For example, as illustrated in FIG. 3, the cartridge 200 has formed therein, a space 210 provided with a reagent or the like, a space 220 provided with containers for storing therein the cell liquid and the reagent delivered by the liquid delivery mechanism 4, and spaces 230 each provided with a culturing vessel 240. Further, these spaces are connected to one another by flow paths, so that various steps related to the cell culture are performed on the inside of the cartridge 200. The cartridge illustrated in FIG. 3 is merely an example. It is possible to arbitrarily set spaces in the cartridge.

For example, when iPS cells are to be cultured, the following procedure is performed in the cartridge 200: separating nucleated cells from liquid (e.g., blood) containing nucleated cells; culturing and expanding CD34 positive cells; introducing an initialization factor; and a culturing process after the introduction. While these steps are performed, the cartridge 200 is switched between the state illustrated in the top section of FIG. 2 and the state illustrated in the bottom section thereof in accordance with the steps. In other words, at the steps of delivering the cell liquid or the reagent and switching between the flow paths, the cartridge 200 is in the state illustrated in the top section of FIG. 2 (the state in which the cartridge 200 has been ejected from the incubator 2). At the step of performing a culturing process, the cartridge 200 is in the state illustrated in the bottom section of FIG. 2 (the state in which the cartridge 200 is placed inside the incubator 2).

Although not illustrated in the drawings, the cartridge 200 has formed therewith a supply port used for supplying the ambient air in the chamber space 22 to the spaces 230. Accordingly, when the cartridge 200 has been placed in the chamber space 22, the ambient air in the chamber space 22 is supplied to the spaces 230 due to diffusion via the supply port, so that the culturing vessels 240 are placed under the environment condition controlled in the chamber space 22. Further, the supply port may be provided with a filter for blocking germs, bacteria, and/or the like.

A configuration of the cell culture apparatus 100 has thus been explained. The cell culture apparatus 100 configured as described above makes it possible to maintain the cell culture environment in the incubator. As explained above, in the cell culture apparatus 100, the cartridge 200 may be installed into and ejected from the chamber space 22 of the incubator 2, in accordance with the various steps related to the cell culture. In this situation, in the state where the cartridge 200 has been ejected from the chamber space 22 (i.e., the state in which the cartridge is not present above the moving equipment 6 or the state in which the cartridge has been pulled out of the chamber space 22 for liquid delivery of the like), the ambient air in the chamber space 22 might be mixed with ambient air in the space outside the incubator via the opening 21, and as a result, the environment inside the chamber space 22 might change. In this situation, the space outside the incubator is managed to have temperatures in the range of 20° C. to 25° C., for example.

To cope with the circumstances described above, the cell culture apparatus 100 according to the present embodiment is configured to maintain the cell culture environment in the incubator, with a configuration where the opening is closed regardless of the state of the cartridge 200. More specifically, the cell culture apparatus 100 includes an opening cover configured to open the opening 21 in association with the moving operation of the moving equipment 6 to install the cartridge 200 and/or configured to close the opening in association with the moving operation of the moving equipment 6 to eject the cartridge 200. In the following sections, details of the cell culture apparatus 100 will be explained.

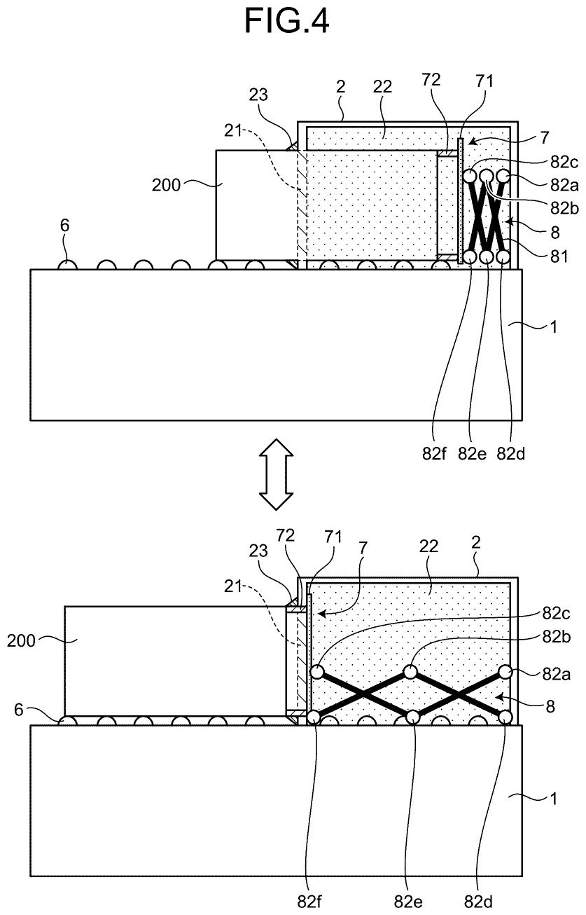

FIG. 4 is a schematic drawing illustrating an exemplary configuration of the cell culture apparatus 100 according to the first embodiment. In the present example, FIG. 4 schematically illustrates the cell culture apparatus 100 as viewed from a direction orthogonal to the direction in which the cartridge 200 is moved by the moving equipment 6. For example, as illustrated in FIG. 4, in the chamber space 22 of the incubator 2, the cell culture apparatus 100 includes an opening cover 7 and a cover supporter 8. The opening cover 7 is an example of an opening cover. The cover supporter 8 is an example of a supporter.

The opening cover 7 includes a cover part 71 and a projection part 72 and is configured, when the cartridge 200 has been ejected from the chamber space 22, to close the opening 21 as illustrated in the bottom section of FIG. 4. More specifically, the opening cover 7 is formed to have such a size that the cover part 71 is able to cover the entirety of the opening 21, while the projection part 72 has a size so as to be fitted with the opening 21. In other words, the opening cover 7 is configured to close the opening 21, as a result of the cover part 71 covering the opening 21 from the inside of the incubator 2, and also, the projection part 72 being fitted with the opening 21.

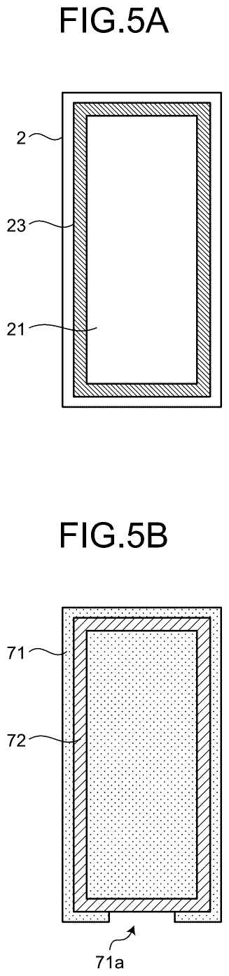

In this situation, as illustrated in FIG. 4, on the outside of the face in which the opening 21 is formed, the incubator 2 is provided with a seal member 23 for the purpose of tightly closing the opening 21. FIG. 5A is a schematic drawing illustrating an example of the seal member 23 according to the first embodiment. FIG. 5A schematically illustrates the incubator 2 as viewed from the opening 21 side. For example, as illustrated in FIG. 5A, on the outside of the face in which the opening 21 is formed, the incubator 2 has the seal member 23 provided along the contour of the opening 21 over the entire perimeter of the opening 21. In this situation, the seal member 23 is an example of a seal.

For example, as a result of the projection part 72 of the opening cover 7 illustrated in FIG. 5B being fitted with the opening 21 of the incubator 2 illustrated in FIG. 5A, the projection part 72 comes into close contact with the seal member 23, so that the opening 21 is tightly closed. In this situation, FIG. 5B illustrates the example in which the projection part 72 is formed so as to project in the manner of a hollow square surrounding a center part along the contour of the opening 21; however, possible embodiments are not limited to this example. A projection part may be formed so as to project in the same shape as the opening 21. Further, FIG. 5B is a schematic drawing illustrating the example of the opening cover 7 according to the first embodiment and schematically illustrates the opening cover 7 as viewed from the opening 21 side.

As explained above, in association with the operation of the moving equipment 6 to move the cartridge 200, the opening cover 7 is configured to open and close the opening 21. In this situation, the position of the opening cover 7 relative to the opening 21 is changed by the cover supporter 8 configured to move in conjunction with the moving operations of installing and ejecting the cartridge 200. In other words, in the cell culture apparatus 100, the opening cover 7 is moved by the cover supporter 8 configured to move in conjunction with the operations to move the cartridge 200, so that the opening 21 is opened and closed. In this situation, the cover part 71 of the opening cover 7 has a cutout part 71a in a lower part, as illustrated in FIG. 5B. In other words, the cover part 71 has formed therein the cutout part 71a for avoiding interference with the moving equipment 6.

Returning to the description of FIG. 4, the cover supporter 8 may be a linking mechanism, for example, and includes linking parts 81 and joint parts 82a to 82f. However, the cover supporter 8 illustrated in FIG. 4 is merely an example. The quantities of the linking parts and the joint parts are not limited to those illustrated in the drawing.

For example, in the cover supporter 8, the linking parts 81 are coupled with the joint parts 82a to 82f so as to be rotatable on the joint parts 82a to 82f serving as axes. In this situation, the joint part 82a is supported on an inner wall of the incubator 2, so as to be able to linearly move along the vertical direction. Further, the joint part 82c is supported so as to be able to linearly move along the vertical direction, on the face of the cover part 71 opposite from the face on which the projection part 72 is formed. Further, the joint part 82d is fixed to the inner wall of the incubator 2. Further, the joint part 82f is fixed to the face of the cover part 71 opposite from the face on which the projection part 72 is formed.

For example, when the moving equipment 6 carries out the moving operation of inserting the cartridge 200 into the chamber space 22, due to the force of the cartridge 200 moving to the inside of the chamber space 22, the opening cover 7 is pressed toward the interior of the chamber space 22, and also, the joint part 82a and the joint part 82c slide and move upward, so that the cover supporter 8 contracts in the moving direction of the cartridge 200. As a result, the opening cover 7 that has so far closed the opening 21 now opens the opening 21. In this situation, because the shape of the cross-section of the cartridge 200 orthogonal to the moving direction is substantially identical to the shape of the opening 21, the opening 21 is substantially maintained in the closed state during the operations of moving the cartridge 200. In the state where at least a part of the cartridge 200 is installed in the chamber space 22, the opening 21 is closed by the cartridge 200.

In contrast, when the moving equipment 6 carries out the moving operation of pulling out the cartridge 200 from the inside of the chamber space 22, the cover supporter 8 is released from the force in the contracting direction, so that the joint part 82a, the joint part 82b, and the joint part 82c move downward due to the weight thereof. Consequently, as illustrated in the bottom section of FIG. 4, the cover supporter 8 expands in the moving direction of the cartridge 200. Consequently, the opening 21 is closed, as a result of the cover part 71 of the opening cover 7 covering the opening, and also, the projection part 72 being fitted with the opening 21.

As explained above, the cell culture apparatus 100 according to the present embodiment is configured, so that the opening cover 7 closes the opening 21 while the cartridge 200 is in the ejected state and so that the cartridge 200 closes the opening 21 while the cartridge 200 is being moved into the incubator 2 and while the cartridge 200 is in the state of being installed in the chamber space 22. With this configuration, the cell culture apparatus 100 is able to prevent the ambient air in the chamber space 22 from mixing with the ambient air outside the incubator 2 and is thus able to maintain the cell culture environment in the incubator.

### Modification Examples

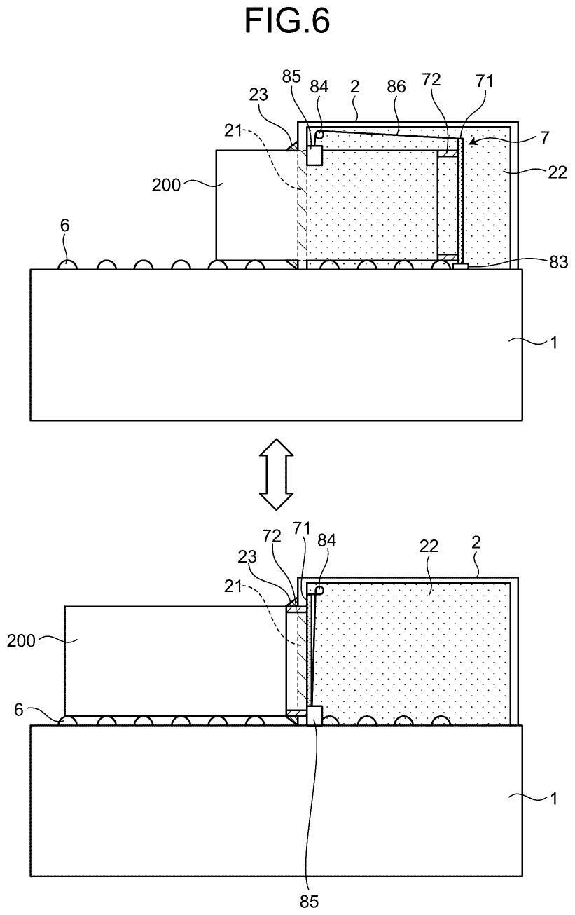

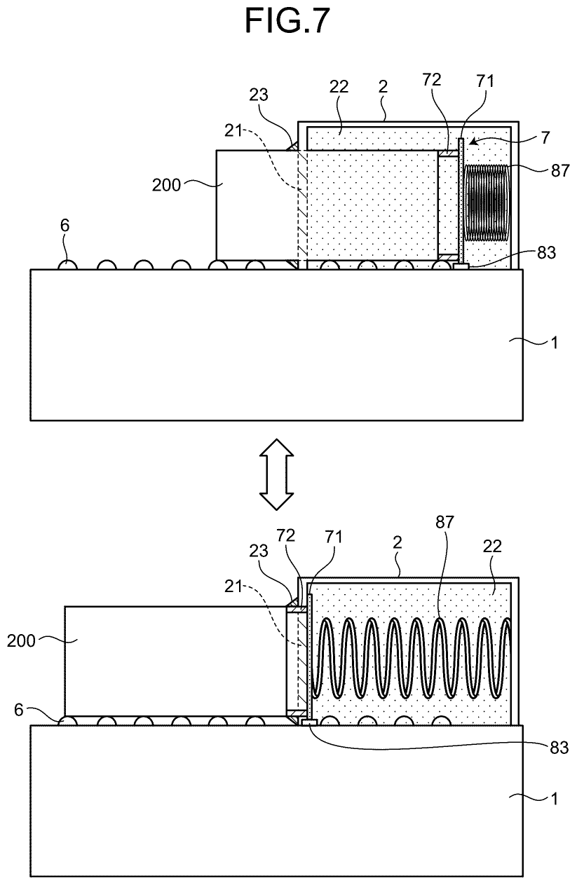

In the above embodiment, the example was explained in which the linking mechanism is used as the cover supporter 8; however, possible embodiments are not limited to this example. It is also acceptable to realize the cover supporter 8 by using other methods. In the following sections, other examples of the cover supporter 8 will be explained, with reference to FIGS. 6 and 7. FIGS. 6 and 7 are schematic drawings illustrating exemplary configurations of the cell culture apparatus according to modification examples. FIGS. 6 and 7 schematically illustrate the cell culture apparatus 100 as viewed from a direction orthogonal to the direction in which the cartridge 200 is moved by the moving equipment 6.

For example, in the cell culture apparatus 100 according to a modification example, as illustrated in FIG. 6, the cover supporter 8 includes a sliding supporting unit 83, a pulley 84, a weight 85, and a wire 86. The sliding supporting unit 83 is configured to support the opening cover 7 so as to be slidable along the moving direction of the cartridge 200. In other words, the sliding supporting unit 83 is configured to support the opening cover 7 so as to be able to slide and move in left-and-right directions in the drawing. The pulley 84 is configured to transmit tension applied to the wire 86 connecting the opening cover 7 to the weight 85. The weight 85 is connected to the opening cover 7 via the wire 86.

For example, when the moving equipment 6 carries out the moving operation of inserting the cartridge 200 into the chamber space 22, due to the force of the cartridge 200 moving to the inside of the chamber space 22, the opening cover 7 supported by the sliding supporting unit 83 moves as being pressed toward the interior of the chamber space 22, and also, tension is applied to the wire 86 and the weight 85 is lifted. As a result, as illustrated in the top section of FIG. 6, the opening cover 7 that has so far closed the opening 21 now opens the opening 21.

In contrast, when the moving equipment 6 carries out the moving operation of pulling out the cartridge 200 from the inside of the chamber space 22, a space is formed between the cartridge 200 and the projection part 72. In this situation, because the opening cover 7 is connected to the weight 85 via the wire 86, tension is no longer applied to the weight 85, and the weight 85 starts descending. When the weight 85 has descended, tension in the opposite direction is applied to the wire 86, so that the opening cover 7 moves toward the opening 21 side. Consequently, as illustrated in the bottom section of FIG. 6, the opening 21 is closed, as a result of the cover part 71 of the opening cover 7 covering the opening, and also, the projection part 72 being fitted with the opening 21.

Further, for instance, in the cell culture apparatus 100 according to another modification example, as illustrated in FIG. 7, the cover supporter 8 includes the sliding supporting unit 83 and a spring 87. The sliding supporting unit 83 is configured to support the opening cover 7 so as to be slidable along the moving direction of the cartridge 200. One end part of the spring 87 is fixed on an inner wall of the incubator 2, whereas the other end part thereof is fixed to the face of the cover part 71 opposite from the face on which the projection part 72 is formed.

For example, when the moving equipment 6 carries out the moving operation of inserting the cartridge 200 into the chamber space 22, due to the force of the cartridge 200 moving to the inside of the chamber space 22, the opening cover 7 supported by the sliding supporting unit 83 moves as being pressed toward the interior of the chamber space 22, and also, the spring 87 is compressed. As a result, as illustrated in the drawing in the top section of FIG. 7, the opening cover 7 that has so far closed the opening 21 now opens the opening 21.

In contrast, when the moving equipment 6 carries out the moving operation of pulling out the cartridge 200 from the inside of the chamber space 22, a space is formed between the cartridge 200 and the projection part 72. In this situation, because the opening cover 7 has the compressed spring fixed thereto, elastic force of the spring is applied thereto, so that the opening cover 7 moves toward the opening 21 side. Consequently, as illustrated in the bottom section of FIG. 7, the opening 21 is closed, as a result of the cover part 71 of the opening cover 7 covering the opening, and also, the projection part 72 being fitted with the opening 21.

As explained above, according to the first embodiment, while the cartridge 200 has the culturing vessels 240 used for culturing the cells, the incubator 2 forms the chamber space 22 in which at least a part of the cartridge 200 including at least one of the culturing vessels 240 is installed. The opening 21 is configured to allow communication between the inside and the outside of the chamber space 22 and is provided for the purpose of installing at least a part of the cartridge 200 in the chamber space 22. The moving equipment 6 is configured to move the cartridge 200 for the purpose of installing or ejecting at least a part of the cartridge 200 into or from the chamber space 22 via the opening 21. The opening cover 7 is configured to open the opening 21 in association with the moving operation of the moving equipment 6 to install the cartridge 200 and/or configured to close the opening 21 in association with the moving operation of the moving equipment 6 to eject the cartridge 200. Consequently, the cell culture apparatus 100 according to the first embodiment is able to prevent the ambient air in the chamber space 22 from mixing with the ambient air in the external space and to thus make it possible to maintain the cell culture environment in the incubator 2.

Further, according to the first embodiment, the position of the opening cover 7 relative to the opening 21 is changed by the cover supporter 8 configured to move in conjunction with the moving operations of installing and ejecting the cartridge 200. Consequently, the cell culture apparatus 100 according to the first embodiment makes it possible to control the opening and closing of the opening 21 realized by the opening cover 7, in conjunction with the operations to move the cartridge.

Further, according to the first embodiment, in the state where at least a part of the cartridge 200 is installed in the chamber space 22, the opening 21 is closed by the cartridge 200. Consequently, the cell culture apparatus 100 according to the first embodiment is able to close the opening 21 with the cartridge 200 itself and to thus make it possible to easily maintain the cell culture environment in the incubator 2.

In addition, according to the first embodiment, during the operations of moving the cartridge 200, the opening 21 is substantially maintained in the closed state. Consequently, the cell culture apparatus 100 according to the first embodiment is able to keep the chamber space 22 as a closed space at all times and to thus make it possible to maintain the cell culture environment in the incubator 2.

Furthermore, according to the first embodiment, the seal member 23 is configured to tightly close the opening 21. Consequently, the cell culture apparatus 100 according to the first embodiment is able to cause the chamber space 22 to be the closed space with a higher level of precision and to thus make it possible to maintain the cell culture environment in the incubator 2.

### Second Embodiment

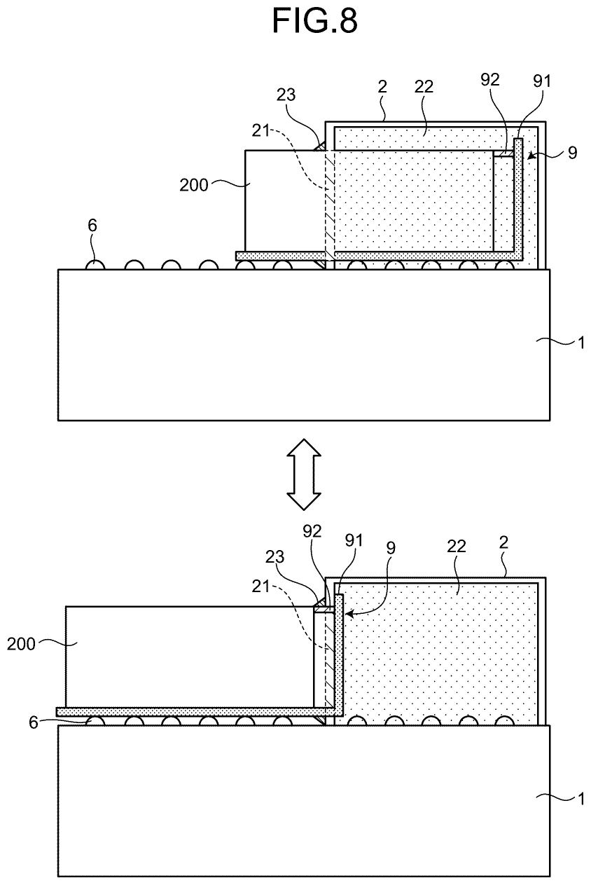

In the first embodiment described above, the example was explained in which the opening cover 7 is supported by the cover supporter 8. In a second embodiment, an example will be explained in which the opening cover 7 is formed in a holding equipment configured to hold the cartridge 200. FIG. 8 is a schematic drawing illustrating an exemplary configuration of a cell culture apparatus according to the second embodiment. In this situation, the cell culture apparatus 100 according to the second embodiment is different from that of the first embodiment for including the holding equipment, in place of the cover supporter 8. In the following sections, this feature will primarily be explained. In the present example, FIG. 8 schematically illustrates the cell culture apparatus 100 as viewed from a direction orthogonal to the direction in which the cartridge 200 is moved by the moving equipment 6.

As illustrated in FIG. 8, the cell culture apparatus 100 according to the second embodiment includes a holding equipment 9 configured to hold the cartridge 200. The holding equipment 9 has the cartridge 200 placed thereon and is moved by the moving equipment 6. In other words, the moving equipment 6 according to the second embodiment is configured to carry out the moving operation of installing the cartridge 200 and the moving operation of ejecting the cartridge 200, by moving the holding equipment 9 configured to hold the cartridge 200. In this situation, the holding equipment 9 is an example of a holding unit.

In this situation, the holding equipment 9 is provided with an opening cover. More specifically, as illustrated in FIG. 8, the holding equipment 9 is provided with a cover part 91 configured to cover the opening 21 and a projection part 92 configured to be fitted with the opening 21. As illustrated in FIG. 8, in the second embodiment, the bottom face of the holding equipment 9 is formed so as to be in close contact with the seal member 23 provided on the lower end side of the opening 21.

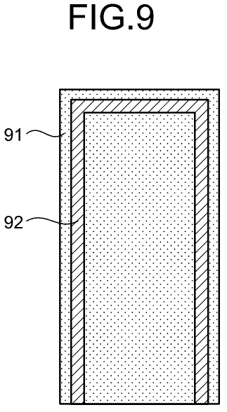

FIG. 9 is a schematic drawing illustrating an example of the opening cover according to the second embodiment. FIG. 9 schematically illustrates the opening cover as viewed from the opening 21 side. As illustrated in FIG. 9, in the opening cover according to the second embodiment, the cover part 91 has formed therewith the projection part 92 having a U-shape. Further, the cover part 91 is formed in such a manner that the face of the cover part 91 positioned at the lower end in FIG. 9 is on the same plane as the bottom face of the holding equipment 9.

In this situation, the length, in terms of a short-axis direction, of a holder (a flat plate member on which the cartridge is placed) of the holding equipment 9 to hold the cartridge is configured to be equal to the length of the projection part 92 in the short-axis direction. In other words, the holder of the holding equipment 9 is arranged to be shorter, in the short-axis direction, than the length of the cover part 91 in the short-axis direction illustrated in FIG. 9. Further, an end part, in terms of the short-axis direction, of the holder of the holding equipment 9 is formed to be on the same plane as the plane on which an end part, in terms of the short-axis direction, of the projection part 92 is positioned. With these arrangements, the holding equipment 9 is configured so that only the holder holding the cartridge 200 is ejected to the outside of the chamber space 22.

For example, when the moving equipment 6 has carried out the moving operation of inserting the holding equipment 9 into the chamber space 22 (the moving operation of inserting the cartridge 200 into the chamber space 22), the opening cover formed in the holding equipment 9 moves as being pressed toward the interior of the chamber space 22 so that, as illustrated in the top section of FIG. 8, the opening cover that has so far closed the opening 21 now opens the opening 21.

In contrast, when the moving equipment 6 has carried out the moving operation of pulling out the holding equipment 9 from the chamber space 22 (the moving operation of pulling out the cartridge 200 from the inside of the chamber space 22), the opening cover moves toward the opening 21 side. Consequently, as illustrated in the bottom section of FIG. 8, the opening 21 is closed, as a result of the cover part 91 of the opening cover covering the opening, and also, the projection part 92 being fitted with the opening 21.

As explained above, according to the second embodiment, the opening cover is provided for the holding equipment 9 configured to hold the cartridge 200. By moving the holding equipment 9, the moving equipment 6 is configured carry out the moving operation of installing the cartridge 200 and the moving operation of ejecting the cartridge 200. Consequently, the cell culture apparatus 100 according to the second embodiment makes it possible to easily realize the configuration of the opening cover.

As explained above, at least one aspect of the embodiments described above makes it possible to maintain the cell culture environment in the incubator.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A cell culture apparatus comprising:
a casing that, while a cartridge has a culturing vessel used for culturing cells, forms a chamber space in which at least a part of the cartridge including the culturing vessel is installed;
an opening configured to allow communication between an inside and an outside of the chamber space and provided for a purpose of installing said at least a part of the cartridge in the chamber space;
a moving equipment configured to move the cartridge, for a purpose of installing or ejecting said at least a part of the cartridge into or from the chamber space via the opening; and
an opening cover configured to open the opening in association with a moving operation of the moving equipment to install the cartridge and/or configured to close the opening in association with a moving operation of the moving equipment to eject the cartridge.

2. The cell culture apparatus according to claim 1, wherein a position of the opening cover relative to the opening is changed by a supporter configured move in conjunction with the moving operations of installing and ejecting the cartridge.

3. The cell culture apparatus according to claim 1, wherein, in a state where said at least a part of the cartridge is installed in the chamber space, the opening is closed by the cartridge.

4. The cell culture apparatus according to claim 1, wherein, during the operations of moving the cartridge, the opening is substantially maintained in a closed state.

5. The cell culture apparatus according to claim 1, wherein
the opening cover is provided for a holder configured to hold the cartridge, and
the moving equipment is configured to carry out the moving operation of installing the cartridge and the moving operation of ejecting the cartridge, by moving the holder.

6. The cell culture apparatus according to claim 1, further comprising: a seal provided for a purpose of tightly closing the opening.

7. A cell culture system that includes a cartridge having a culturing vessel used for culturing cells and a cell culture apparatus in which the cartridge is placed, wherein
the cell culture apparatus comprises:
a casing that forms a chamber space in which at least a part of the cartridge including the culturing vessel is installed;
an opening configured to allow communication between an inside and an outside of the chamber space and provided for a purpose of installing said at least a part of the cartridge in the chamber space;
a moving equipment configured to move the cartridge, for a purpose of installing or ejecting said at least a part of the cartridge into or from the chamber space via the opening; and
an opening cover configured to open the opening in association with a moving operation of the moving equipment to install the cartridge and/or configured to close the opening in association with a moving operation of the moving equipment to eject the cartridge.
